(19) United States
(12) Patent Application Publication
(10) Pub. No.: US 2021/0275835 A1
(43) Pub. Date: Sep. 9, 2021

(54) DEVICE FOR INDUCING EXFOLIATION OF CELLS AND/OR TISSUE FRAGMENTS FOR ENHANCED CYTOPATHOLOGIC CELL COLLECTION

(71) Applicant: Adenocyte Ltd., Beit Shemesh (IL)

(72) Inventor: Dan Adam, Haifa (IL)

(73) Assignee: Adenocyte Ltd., Beit Shemesh (IL)

(21) Appl. No.: 17/007,953

(22) Filed: Aug. 31, 2020

Related U.S. Application Data
(60) Provisional application No. 62/893,463, filed on Aug. 29, 2019.

(51) Int. Cl. A61N 7/00

(52) U.S. Cl. CPC A61N 7/00; A61N 2007/0078; A61N 2007/0073; A61N 2007/0004

(57) ABSTRACT

An ultrasound probe configured for insonating a pancreas. The probe is sized and shaped to be placed on a patient's abdomen in an area that is just between the right and left rib cage sections. The probe transmits ultrasound energy in order to insonate substantially the entire pancreas at the same time. One or more cavitation detectors are positioned within the probe to detect an incidence of cavitation.

### RELATED APPLICATIONS

This application claims the benefit of pending U.S. Prov. Appl. 62/893,463 filed Aug. 29, 2019, the contents of which are incorporated by reference herein.

### FIELD OF THE INVENTION

The invention relates to the field of medical devices, more specifically, to an ultrasound probe configured to insonate an entire organ in order to induce exfoliation of cells and tissue.

### BACKGROUND OF THE INVENTION

Over 95% of cancers in adults are carcinomas, meaning that they originate in the epithelial lining of an organ. In many cases these epithelia are in direct contact with a surrounding body fluid into which the epithelial cells are continually shed as part of the natural process of epithelial regeneration. An example are pancreatic ductal cells that are continually being shed into the pancreatic juice, lung cells that are continually shed into the sputum, and bladder cells that are continually being shed into the urine. Dysplastic and cancer cells that may be present in the subject epithelium are also naturally shed into the surrounding fluid along with normal epithelial cells. In many cases these body fluids are accessible to non-invasive or minimally invasive sampling methods. Exfoliated cells found in these sampled fluids can then be concentrated using standard laboratory techniques for subsequent microscopic evaluation by a cytopathologist allowing, at least in theory, for the early detection of dysplasia and cancer. While cytopathologic examination of cells exfoliated into surrounding body fluids generally has high specificity, its sensitivity for the detection of dysplasia and cancer is often limited by the fact that the slow rate of natural exfoliation of epithelial cells into the surrounding fluid results in a very small sample with few cells for the pathologist to examine. This currently limits the cytopathologic detection of dysplasia and cancer in: the mediastinum, the pleura, the pericardium, the peritoneum, the lung, the breast, the salivary glands, the meninges, the pancreatic ducts, pancreatic cysts, the kidney, the liver, the bladder, and the ovaries among others.

For example, the pancreas is comprised of a variety of cell types, each of which may give rise to a different type of cancer. Pancreatic ductal adenocarcinoma (PDAC) is the fourth leading cause of cancer-related death in the United States. This is due to its often late diagnosis at metastatic stages, its aggressive biology, and only partial response to known chemotherapies. Cytological evaluation of pancreatic juice has been shown to have a high-sensitivity, upwards of 79% in diagnosing pancreatic cancer.

Because there are usually few or no early symptoms, pancreatic cancer is often advanced by the time it is discovered. For this reason, by 2030, pancreatic cancer is projected to be the second leading cause of cancer death in the United States (1).

Pancreatic screening and surveillance is performed for patients at increased risk for developing pancreatic cancer because of their pancreatic cancer family history, a pancreatic cancer susceptibility gene mutation, or having incidentally detected pancreatic cysts.

Current tests for pancreatic cancer include endoscopic ultrasonography (EUS) and endoscopic magnetic resonance imaging/magnetic resonance cholangiopancreatography (ERCP). Although these tests are accurate for detecting pancreatic cysts (2, 3, 4) they are not well suited for detecting small solid pancreatic cancers, as evidenced by the number of patients who develop pancreatic cancer despite regular surveillance. This reflects missed opportunities for early detection (5).

Cytopathology, an essential clinical criterion in the diagnosis of disease, has been tested as a diagnostic modality for pancreatic cancer. For example, in 1974, Yoshihiko Endo and coworkers first reported the cytodiagnosis of pancreatic cancer from the collection of pancreatic juice with a duodenoscope (6). They reported a sensitivity of 79%. Through the subsequent decades, many researchers have attempted to use cytologic examination of pancreatic juice to determine pancreatic malignancy, but sensitivity ranges remained stubbornly around 70-80% (7, 8). The shortfall in sensitivity observed from multiple endoscopists through multiple years likely reflect shortcomings in typical cell collection and analysis methods. In addition, low levels of cells or tissue fragments in fluid samples obtained for histological analysis in bioassays is a problem that exists for multiple bioassay types.

Thus, there is a need for increasing the yield of cells exfoliated from various organs and tissue types for collection and subsequent analysis in order to enhance the specificity of such diagnostic tests. There also is a need to induce the exfoliation of intact tissue, which may be used to provide an enface view of a tissue segment. In regard to pancreatic cancer in particular, there is a need for improved cellular collection and tissue fragment collection techniques for detecting abnormal cells from a background of normal cells in the pancreas.

### SUMMARY OF THE INVENTION

Embodiments of the invention set forth herein provide devices and systems for obtaining cells, and/or tissue fragments and other molecules at a much greater rate than previously observed by different means.

The invention includes an ultrasound transducer that is designed to deliver sufficient ultrasound radiation to a target organ in order to impart sufficient energy to the tissue of the target organ to induce exfoliation of cells and/or tissue. In embodiments, the ultrasound transducer is configured to insonate an entire organ or the majority of an organ, substantially simultaneously.

The transducer or probe according to embodiments of the invention includes one or more straps that are utilized to selectively affix the probe to a patient and maintain the probe securely in place.

Still in other embodiments of the invention, the probe includes a cavitation detector that terminates application of radiation or lowers a dosage of radiation in response to the detection of cavitation.

In an embodiment of the invention, the probe is used as part of a method of inducing exfoliation of pancreatic cells or tissue and collecting pancreatic juice that includes such exfoliated cells or tissue for pathologic analysis. In an embodiment, the method includes administering an ultrasound contrast agent that forms microbubbles in a patient's circulatory system prior to insonating the subject. After introducing microbubbles, the organ or tissue, such as a pancreas, is subjected to wide area ultrasound energy provided by the transducer. The ultrasound application of embodiments of the invention may be described as Low Intensity Non-Focused Ultrasound (LINFU). In embodiments of the invention, the ultrasound energy combined with the energy exerted by the microbubbles causes pancreatic cells and, optionally, tissue fragments to disassociate and/or exfoliate. The patient, in the case of obtaining a pancreas sample, is subsequently injected with secretin, a drug that induces pancreatic secretion. In this regard, some of the exfoliated and dislodged cells and tissue fragments may be deposited into the pancreatic juice, which is then collected endoscopically. The cells and/or tissue fragments in the obtained enriched samples are then analyzed morphologically and/or using molecular biomarkers to detect a presence or absence of cellular abnormality. Microbubbles, which may be used together with ultrasound insonation to enhance ductal epithelial cell exfoliation, may also be generated locally by high enough ultrasound intensity. Here, this process may be enhanced by employing a non-symmetrical waveform, produced by concurrent transmitting a plurality of different frequencies (e.g., 400 kHz, 800 kHz and 1600 kHz), where the phases of each transmitted frequency are synchronized to produce coronal planes, parallel to the body/probe surface, of superposition of negative (rarefaction) waves. Since ductal fluids and blood are less viscous than tissue, microbubble will be formed in these fluids at lower insonation intensities than those needed to generate microbubbles within tissue. In regard to pancreatic ductal fluids, they contain high concentration of bicarbonate, thus generation of microbubbles will be even more enhanced, allowing for much lower insonation intensities than those that may produce microbubbles within the tissue.

The probe used in combination with these procedures can dramatically increase the total number of cells to be expressed in the pancreatic juice. Moreover, the probe used in conjunction with exemplary procedures set forth herein may induce the separation of intact tissue fragments from the pancreas.

The probe used in combination with these procedures, when applied to the other organs or body sites, such as, the mediastinum, pleura, pericardium, peritoneum, lung, breast, salivary glands, meninges, pancreatic ducts, pancreatic cysts, kidney, liver, bladder, or ovaries, may dramatically increase the total number of cells to be expressed in the surrounding fluids of such organs. Additionally, the procedures set forth herein may additionally induce the separation of intact tissue fragments from the above-cited exemplary organs. For example, the probe set forth herein may be utilized to induce exfoliation of lung cells into surrounding sputum or to induce to the exfoliation of bladder cells and bladder tissue into surrounding urine.

### BRIEF DESCRIPTION OF THE DRAWINGS

Illustrative embodiments of the invention will hereafter be described with reference to the accompanying drawings, wherein like numerals denote like elements.

FIG. 1 is a front perspective view of a probe according to an embodiment of the invention.

FIG. 2 is rear perspective view of the probe of FIG. 1.

FIG. 3 is a top perspective view of the probe of FIG. 1, schematically illustrating the direction of ultrasound signals.

FIG. 4 is a front view of the probe of FIG. 1 shown as applied to a patient.

FIG. 5A is a front view of a substantially circular probe in accordance with exemplary embodiments of the invention.

FIG. 5B is a front view of a substantially trapezoidal probe in accordance with exemplary embodiments of the invention

FIG. 6A is a schematic view of a probe configuration in exemplary embodiments of the invention.

FIG. 6B is a schematic view of a probe configuration in exemplary embodiments of the invention.

FIG. 7 is a schematic view of a probe configuration according to an exemplary embodiment of the invention.

FIG. 8 is a schematic representation of exemplary frequencies emitted by individual transmitter elements according to an embodiment of the invention.

FIG. 9 is a schematic representation of a system architecture according to an exemplary embodiment of the invention.

FIG. 10 is an illustration of hardware components forming a system in accordance with an exemplary embodiment of the invention.

### DETAILED DESCRIPTION OF THE INVENTION

Embodiments of the present invention will now be described with reference to the above-identified Drawings. However, the Drawings and the description herein are not intended to limit the scope of the claims. It will be understood that various modifications of the present description are possible without departing from the spirit of the invention. Also, features described herein may be omitted, additional features may be included, and/or features described herein may be combined in a manner different from the specific combinations recited herein, all without departing from the spirit of the invention.

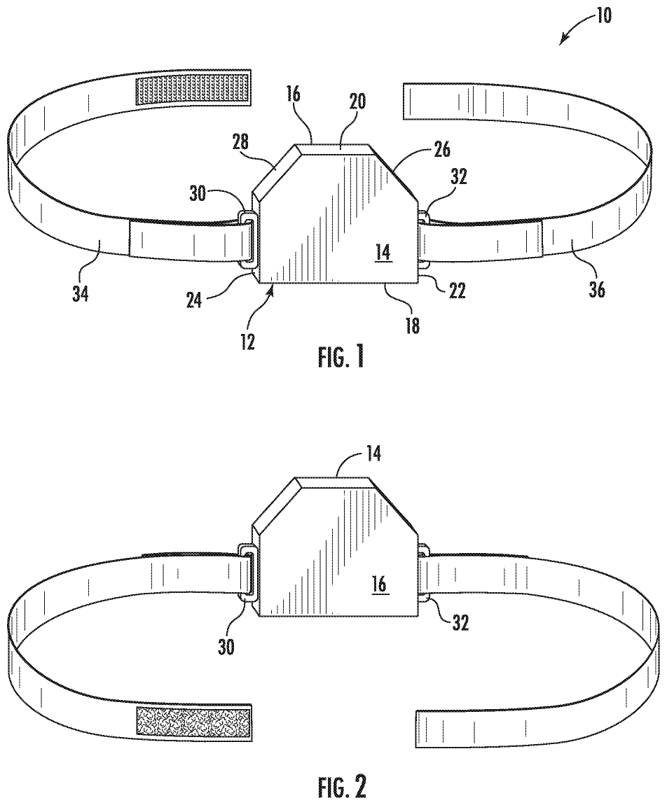

FIG. 1 is a front view of an ultrasound transducer or probe 10 according to an exemplary embodiment of the invention. Probe 10 is formed of one or more piezoelectric elements housed within an outer casing 12. In embodiments of the invention, the probe casing (and transducer head within the casing) is shaped and configured to fit in the external region of a human body below the xiphoid process of the sternum and between the right and left sections (i.e. “lobes”) of the ribcage. In this manner, probe 10 is able to achieve maximum projection of ultrasound energy without the energy being obstructed and/or absorbed by the bones of the rib cage.

As shown, outer casing 12 has a front surface 14, a rear (patient-facing) surface 16 a lower wall 18 and an upper wall 20. A right side wall 22 and a left side wall 23 extend upwardly from lower surface 18. In embodiments of the invention, a first angled wall 26 and a second angled wall 28 bridge the right and left side walls 22, 24 to top surface 20. As shown, top surface is shorter in width than bottom surface, such that angled walls 26, 28 slope toward the centerline of probe 10. In embodiments of the invention, the slope angles of angled walls 26, 28 are configured to approximate the angles of inner rib cage lobes of average-sized adults. For example, in embodiments of the invention, probe 10 has a section that substantially conforms to an external body in an area within the subcostal angle of an average adult rib cage. In this regard, the probe, placed on an abdomen of a patient, is aligned such that ultrasound energy emitted therefrom will enter the patient's body below the ribs.

FIG. 2 shows a rear view of probe 10. Rear surface 16 of probe 10 is configured to contact an external surface of a patient during use.

In embodiments of the invention, right and left walls 22, 24 are provided with an attachment point for a strap. For example, in an embodiment, loops (e.g. 30, 32) are provided on sidewalls 22, 24 for receiving straps 34, 36. In embodiments of the invention, straps 34, 36 are provided with adjustable fasteners, such as for example, Velcro strips. In embodiments of the invention adjustable fasteners are front facing such that a clinician may tighten the straps from a frontal approach.

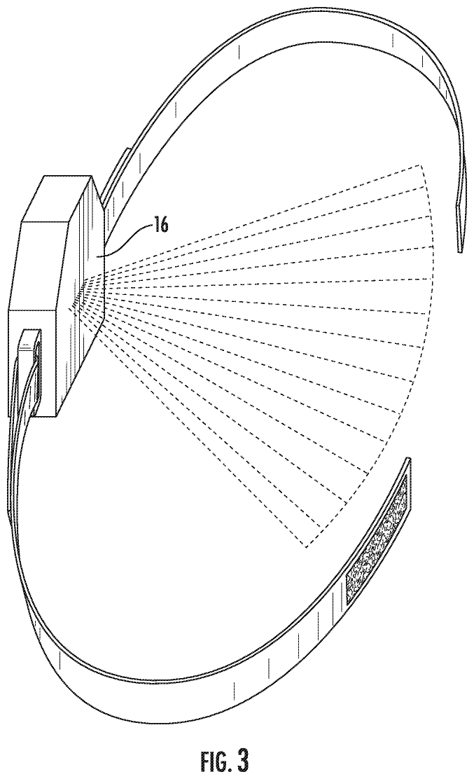

FIG. 3 shows a top perspective view of transducer 10 schematically illustrating the direction of ultrasound pulses emitted from the transducer.

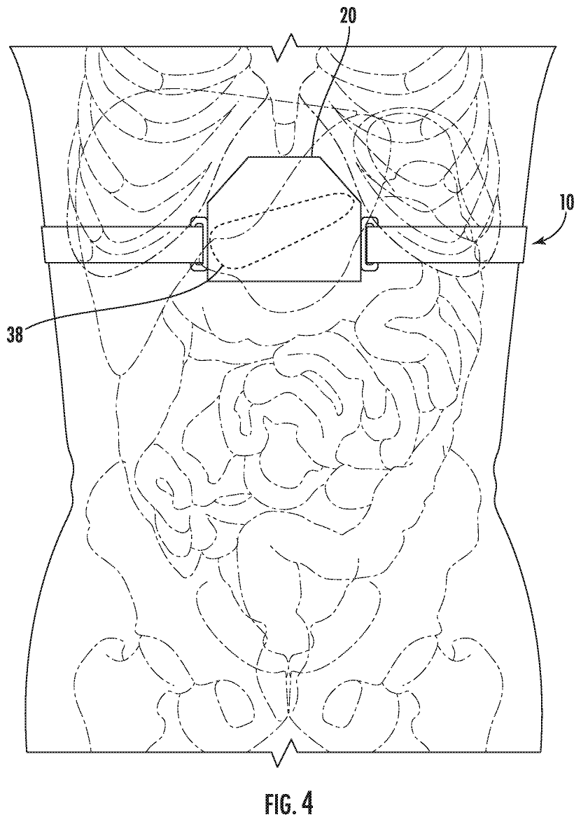

FIG. 4 shows a probe 10 applied to a patient. As shown, the probe 10 is positioned such that top surface 20 is positioned just below the patient's sternum and angled side walls ensure that all areas of the transducer remain between the lobes of the patient's ribcage.

In embodiments of the invention, the probe shape takes advantage of the upper surface area between the patient's rib cage that is not blocked by skeletal structures. With the probe 10 so positioned between the right and left lobes of the patient, the probe 10 is substantially aligned with the patient's pancreas (shown schematically as 38). In this manner, application of ultrasound energy by the probe achieves insonation of substantially the entire pancreas simultaneously.

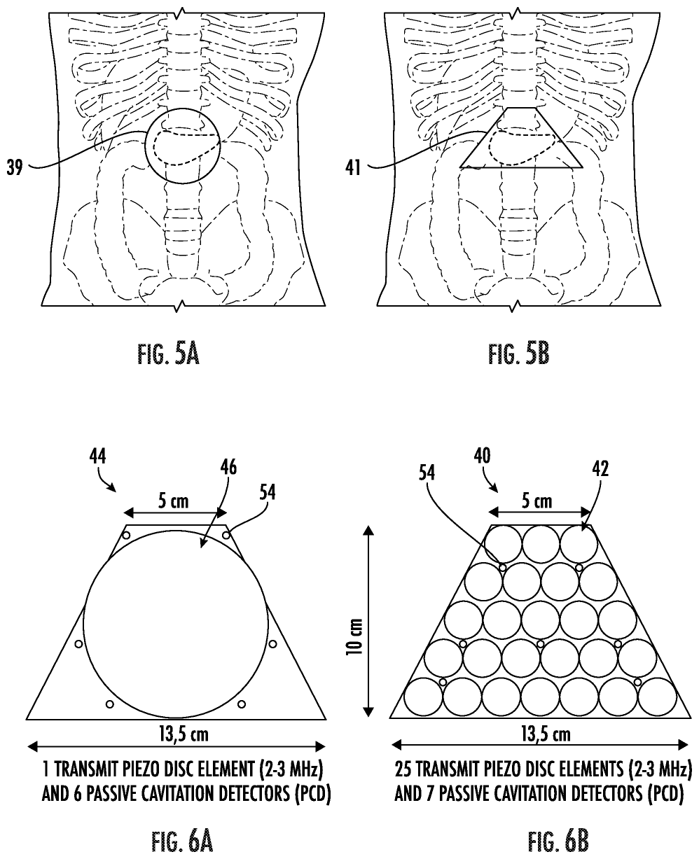

It will be understood by those of ordinary skill in the art that probes may be formed of any of various shapes, sized and configurations in different embodiments of the invention. It will be understood that the size and shape of the probe is optimized to fit project maximum amount of ultrasound energy and be positionable on a patient such that ultrasound beams are unobstructed by bones. For example, FIG. 5A shows a transducer 39 that is substantially round. FIG. 5B shows a substantially trapeziodal transducer 41 formed of an upper wall that is smaller in width than a lower wall, whereby sloping walls bridge the upper and lower walls. It will be understood that the invention utilizes a housing having curved, or sloped aspects that allow for placement on an external body site between the lobes of a patient's ribcage.

It will be understood by those of ordinary skill in the art that probes of the invention may be formed of a singular element or of multiple elements. For example, in an embodiment of the invention, a probe is formed of multiple piezoelectric discs. In the exemplary embodiment shown in FIG. 6 (right image), a probe 40 is formed of 25 piezoelectric discs 42 each having a roughly 16 mm diameter and a frequency range of 2 MHz to 3 MHz. In this regard, a disc having a 1 mm thickness provides a frequency of about 2 MHz (e.g. when formed using PIC255 material, as commercially available from PI Piezo Technology).

FIG. 6 (left image) shows yet another embodiment of the invention 44 where a probe is formed of a single piezoelectric element 46.

It will be understood by those of ordinary skill in the art that any number of piezoelectric elements may be used in different embodiments of the invention. For use in a probe, piezoelectric elements (e.g. 46) are provided with a backing and with impedance matching materials, such as for example a first matching layer and a second matching layer. First and second matching layer provide acoustic impedance matching.

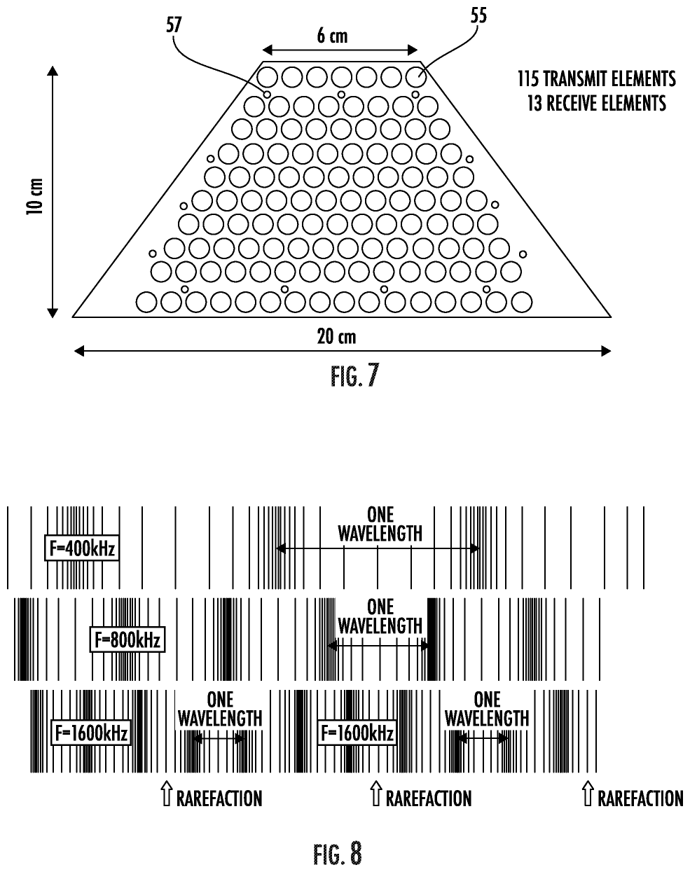

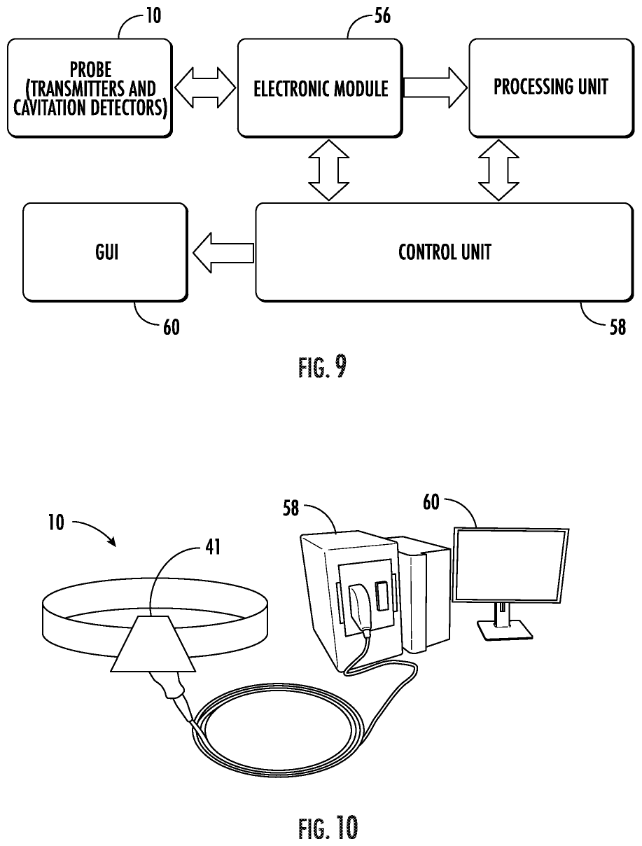

FIG. 7 shows another probe configuration in accordance with an embodiment of the invention. As shown, approximately 115 transmitter elements 55 (e.g. piezoelectric elements) and approximately 13 receiver elements 57 (e.g. cavitation detectors) are provided. In embodiments of the invention, each transmitter element is substantially an 8 mm diameter disc, and respective discs are placed at approximately 9 mm center to center. It will be understood by those of ordinary skill in the art that each of the transmitter elements may be configured to emit the ultrasound beams of the same frequency (e.g. between 2 MHz-4 MHz). In other embodiments, different transmitter elements may be configured to emit ultrasound beams at different frequencies.

In embodiments of the invention, and with reference to FIG. 6, one or more cavitation detectors 54 are provided on the probe to detect the incidence of unstable cavitation. The cavitation detectors 54 may comprise passive cavitation detectors (PCD) or another type of hydrophone. The cavitation detectors 54 are preferably evenly disbursed throughout the probe, for example, as shown in FIG. 6 (small circles). Cavitation detectors provide a safety function by detecting when an ultrasound application is of too high of intensity or duration so as to cause tissue damage. As described more fully below, in the event that cavitation is detected, an associated system automatically runs a remediation routine.

With reference to FIG. 8, in an embodiment of the invention, a probe is configured to emit multiple frequencies in order to produce non-symmetrical waveforms at different locations and times. This may be achieved, for example, by providing transmitter elements configured to emit signals at a plurality of different frequencies (e.g., 400 kHz, 800 kHz and 1600 kHz). Since the production of non-symmetrical waveforms depends of the phases of each transmitted frequency (and the location of the transmitter and its targeted location (i.e. the distance between them)), planes (coronal planes, parallel to the body/probe surface) of superposition of negative (rarefaction) waves are formed, which will be moved forwards and backwards (i.e., farther from and closer to the probe) by shifting the phases among the different frequency generators.

FIG. 9 is a schematic diagram of an exemplary system architecture according to an embodiment of the invention. As shown, an ultrasound probe 10 is electronically coupled to a workstation 56 which controls the output and receives inputs from the probe 10. An integrated or separate control unit 58 is configured to control the functions of the probe 10. A coupled display such as a monitor 60 shows information such as setting and parameter information to an operator.

In embodiments of the invention, the control unit displays to the operator various parameters, including those that the operator is permitted to modify (within the limits pre-set by the system). These may include:
Overall time;
Time of insonation (after contrast agent is injected) before secreting injection, and after secretin injection;
Intensity of insonation;
Frequency of the transmitted waveforms (dependent on the patient's BMI);
Length of burst; and
PRF.

In addition, the control unit receives information from the cavitation detectors, and modifies the intensities accordingly. It may reduce the intensities only in the area where cavitation was detected, or reduce the overall intensities of all transmitters. The control unit also oversees the maintenance of the whole system, including the probe, and the condition of the piezo discs (by measuring their capacitance and resistance).

### Processing Unit:

The processing unit is configured to generate electric signals for activation of the transducers in probe 10. In response to operator input, the processing unit may employ any of different frequencies selected by the operator. It may also allow for the application of single frequencies, concurrent multiple frequencies, different pulse lengths, and control the duration of periods of insonation. In other embodiments, the processing unit may, upon operator input, employ unfocused beams, focused beams to a specific location, or tilted beams (e.g. to insonate organs behind the ribs).

It will be understood that the processing unit is configured to run routines for the optimal performance of a procedure. For example, in embodiments of the invention, one or more software modules are configured to provide prompts and/or instructions to a user at various times during the course of treatment. Prompts may be in the form of an audible signal, such as a beep or a visual signal such as an icon or similar message displayed on an associate display. In embodiments of the invention, the system is configured to prompt a user i) when to start an IV injection (continuous, or boluses) of the contrast agent, ii) when to inject e.g. secretin, iii) when to stop the insonation etc.

In embodiments of the invention, the processing unit receives signals from the cavitation detectors (e.g. 7 detectors). The processing of the data provides information regarding the existence of inertial cavitation, in particular, once a threshold is crossed over. The processing of the data from the multiple detectors provides information (by triangulation and timing) regarding the location of the source of implosions. Multiple sources of inertial cavitation (implosion caused at several locations) can also be detected and registered.

Once inertial cavitation (implosion) is detected, the information is transferred to the control unit, for processing and modification of the intensities of the transmitted pulses.

In a different embodiment, the cavitation detectors are used to measure the existence of inertial cavitation employing the Doppler Effect (e.g. Power Doppler, color Doppler, pulse inversion Doppler, decorrelation Doppler, or interleaving Doppler). In this embodiment, the processing unit will also serve as a function generator and transmitter, emitting via the cavitation detectors the transmit signals necessary for measuring the Doppler Effect in the tissue/pancreas, and as a receiver, receiving the incoming back signals via the cavitation detectors, calculating the Doppler Effect, an causing an “alarm” when such signals surpass a threshold (warning the user that cavitation has occurred).

In another embodiment, the processing unit also receives signals from the transmitters, in a Transmit/Receive configuration, during the time periods the transmission is Off. Using beamforming, the data may provide traditional ultrasound “images.” As is known, the quality and contrast of the images will depend on the number of Transmitters/Receivers, and the level of processing. Multiple temporal acquisitions may be processed to provide information regarding the target organ.

The electronic Transmitter/Receiver should allow transition of long bursts (pulses) of, for example, 0.2-1.0 ms, at pulse repetition frequency (PRF) of 3000 to 6000 repetitions per second (whether from multiple transmitters or only one). An exemplary system capable of performing these tasks is Verasonics Vantage 64LE (128 Tx/64 Rx) with Extended Transmit capabilities.

In embodiments of the invention, the probe in accordance with embodiments of the invention is used to target an organ during a sonication procedure. For example, a patient receiving being treated for pancreatic cancer with a regimen of nab-paclitaxel/gemcitabine may be in infused with such medication. After the initiation of infusion, a probe in accordance with embodiments of the invention is employed to induce cellular sonication of the pancreatic tissue. This makes the pancreatic tissue more permeable and therefore, enhances the uptake of the medication.

Having described the subject matter of the application with regard to specific embodiments, it is to be understood that the description is not meant as a limitation since further modifications and variations may be apparent or may suggest themselves to those skilled in the art. It is intended that the present application cover all such modifications and variations. What is Claimed is:

1. An ultrasound probe, comprising:
a housing comprising a front wall, a rear wall, a right wall, a left wall and a segment that is sized and shaped to fit in an external area of a patient between a right rib cage section and a left rib cage section;
a plurality of piezoelectric elements disposed within the housing; and
a plurality of cavitation detectors disposed within the housing.

2. The ultrasound probe of claim 1, whereby the plurality of piezoelectric elements are each configured to emit ultrasound energy of the same frequency.

3. The ultrasound probe of claim 2, whereby the same frequency is a frequency in a range between 2 MHz-4 MHz.

4. The ultrasound probe of claim 1, whereby respective one of the plurality of piezoelectric elements are configured to emit ultrasound energy of different frequencies.

5. The ultrasound probe of claim 1 further comprising a control unit electrically coupled to the ultrasound probe.

6. The ultrasound probe of claim 5, whereby the control unit is configured to control functions of the ultrasound probe.

7. The ultrasound probe of claim 6, whereby the functions comprise and of duration of emission of ultrasound energy, intensity of ultrasound energy, frequency of transmitted waveforms and length of ultrasound energy bursts.

8. The ultrasound probe of claim 7, whereby the control unit is configured to receive signals from respective ones of the plurality of cavitation detectors and run a remediation routine in response.

9. The ultrasound probe of claim 8, whereby the remediation routine comprises reducing an intensity of ultrasound energy emitted from one or more of the plurality of piezoelectric elements disposed.

10. The ultrasound probe of claim 9, whereby the remediation routine comprises reducing an intensity of ultrasound energy emitted to an area where cavitation was detected.
